(19) United States
(12) Patent Application Publication
(10) Pub. No.: US 2020/0143480 A1
(43) Pub. Date: May 7, 2020

(54) Method and apparatus for anonymously pooling capital and making periodic distributions or dividends to remaining pool particpants based on cohort status and finanacial instrument yield combined with mortality credits associated with the longevity of participants

(71) Applicant: Richard T. Strother, Palm Beach, FL (US)

(72) Inventor: Richard T. Strother, Palm Beach, FL (US)

(21) Appl. No.: 16/350,324

(22) Filed: Nov. 5, 2018

(51) Int. Cl. G06Q 40/08; G16H 50/30

(52) U.S. Cl. CPC G06Q 40/08; G16H 50/30

(57) ABSTRACT

The present invention relates to an administered program consisting of a fixed pool of securitized assets allocated to a fixed population of digitized anonymous pool participants divided into Cohorts based on similar age and attributes. For a period of time, pool survivors receive their own allocation of distribution or dividend and also share pro-rata in a deceased pool member's dividend or distribution (a “Mortality Credit”) until the program's end. The result is an increasing yield investment for surviving pool participants. Modern technology such as digital certificates, smart contracts, encryption technology, electronic fingerprints, voiceprints, retinal scans, facial imaging, DNA testing and block chain and hyper ledger distribution technology will be applied in a sequential and encrypted multi-computer process to maintain administrative purpose and the anonymity of the pool participants whereby there is a guarantee that no pool participant can identify another participant in the same Cohort and/or same pool.

### CROSS-REFERENCE TO RELATED APPLICATION

This application claims priority from Provisional U.S. Application No. 62/707,340, filed on Oct. 30, 2017.

### TECHNICAL FIELD OF THE INVENTION

Longevity Insurance innovation derived by use of Financial Instruments and Biometric Identification in Funding Pools of Capital whereby distributions or dividends are distributed to segmented Cohorts of anonymous individuals who are identified by computer based anonymous biometric parameters, digital parameters and/or and block chain and hyper ledger distribution technology for such a time period that is: (i) associated with type of Pool of Capital, and (ii) the longevity or survival of such anonymous Pool Participants.

### BACKGROUND OF THE INVENTION

The present invention relates to a retirement system that addresses longevity risk of selected individuals having common mortality risks. The individuals anonymously participate in an investment group and the investment is typically grown in an accumulation phase or distributed in scheduled distributions or dividends. Thereafter, payments are distributed to surviving participants at a rate and for a number of periods calculated with respect to the longevity experience and expectations of the participants and the achieved investment returns. The problem of outliving savings has existed for some time, others have tried to solve this problem but have failed. A fresh and different approach combines new technologies with old concepts to create an innovative solution to this problem. The embodiments of this invention combines new technology with an obsolete investment tool in order to raise capital at lower costs, benefiting individuals and governments and providing a type of longevity insurance Thus a new method of anonymous computer based distribution is combined with the old concept of an annuity whereby an investment fund (hereinafter referred to as “Pool” or “Pool of Capital”) is shared by investor-subscribers (hereinafter referred to as participant or “Pool Members”). The shares of the Pool distributions or dividends will increase the Pool yields and payouts as certain Pool Members pass away and a Mortality Credit is thereby generated and divided pro-rata among surviving Pool Members. Thus by agreement, the Pool

members stipulate that should they pre-decease the end of the Pool program or the termination event of the Pool, their share of the distribution or dividends shall be divided pro rata amongst the remaining Pool Member survivors, thereby steadily increasing the Survivors' yield or return on investment as the mortality rate increases over time. Thereby each remaining Pool Member Survivor receives not only the original stated distribution or dividend yield but also receives pro rata a “Mortality Credit” from the deceased Pool Member's share, the increasing yield or payout resulting in a type of longevity insurance and lifestyle assurance against inflation and increasing medical costs. This significantly lowers the risk of underestimating savings needed for retirement or the possibility of outliving such savings. Pool Members who pre decease the termination of the Pool time period in most cases may appoint their original capital investment to beneficiaries, their estate, charitable institutions, and where none so are appointed, it will escheat according to law. A new computer based biometric and algorithmic technology is combined with an old idea. by adding modern computer based algorithms, biometrics and block chain and hyper ledger distribution technology to allow anonymity, there arises a very good way to raise capital at a lower cost and provide longevity insurance and lifestyle maintenance in the face of inflation and increasing medical costs, as people live longer lives due to medical innovations and health awareness (such as the dangers of smoking cigarettes).

### SUMMARY OF THE INVENTION

According to a first embodiment of the invention, there is provided a method for distributing an income stream of payments to a group of participant individuals for an indefinite period associated with the longevity of the participants, the method comprising:

providing one or more computers or one or more computer servers, the one or more computers or one or more computer servers having a processor and memory storage with instructions which when executed by the processor perform predetermined functions;

providing a database stored on the memory storage of the at least one or more computers or one or more computer servers;

in an establishment function phase:

collecting information for storage in the database relating to a plurality of potential participants, the information including mortality criteria;

identifying, by the one or more computers or one or more computer servers, from the information collected, common mortality criteria and/or common expected mortality calculated from some or all of the mortality criteria relating to some or all of the potential participants indicating a substantially equivalent mortality risk;

defining, by the one or more computers or one or more computer servers, from some or all the identified common mortality criteria and/or the common expected mortality, requirements for inclusion of potential participants in one or more substantially homogeneous mortality risk groups;

identifying, by the one or more computers or one or more computer servers, a plurality of participants having a substantially equivalent mortality risk as determined by meeting the requirements for inclusion in the one or more groups;

forming one or more groups of some or all of the identified participants, the matching of the participant in any group or pool fund being selected by the participant, whereby biometric data is collected and participant is assigned a combination of alphanumeric or digital PINs, rendering participant anonymous;

establishing a group investment fund, the investment fund being the aggregation of investment payments received from or on behalf of each participant in the group;

investing some or all of the investment fund in an asset allocation portfolio in accordance with pre-determined investment guidelines intended to grow the investment fund; and

in a distribution phase:

distributing a portion of the investment fund as payments at predetermined distribution periods, the portion being made available for distribution payments at the completion of any period wherein some or all of the longevity risk is transferred to the group of participants and an income stream of payments to the participants is made available for as long as they live or until there is only a predetermined number of survivors remaining from the original group or where a predefined time has elapsed after formation of the group or after the occurrence of another predefined circumstance, such as program termination.

Generally, mortality criteria relates to the potential participants' age, gender and health condition; but may include other criteria considered relevant to expected mortality, including living habits, habitat, or type of employment and the like.

Preferably, in an accumulation phase, the asset

allocation portfolio is invested for a predetermined accumulation period prior to the initiation of the distribution phase. In the event of death in the accumulation phase, the participants may have agreed that a death charge or no charge be applied. Generally, the deducted charge is retained in the investment fund for the benefit of the remaining participants;

In the distribution phase, after life-death verification process by the administrator, payments are generally made only to any surviving participants remaining. However, the participants have agreed that the principal can be made beneficiaries of non-survivors for a predetermined period after the termination of the program criteria have been met;

In the event that the income stream of payments is to be made after the effluxion of predefined time, the time may be determined based on the age of the group or the period since group formation.

Typically some or all of the investment risk associated with the investment fund is transferred to the group of participants;

Generally, the aim is to maintain increasing distribution payments, recognizing the reliance participants may place on the income stream as a hedge against depletion of financial recourses in retirement as a result of longevity or as a hedge against inflation.

Fees may be payable to third parties relating to the formation and/or management of the investment fund and for the calculation and/or management of the distributed income stream of payments. These fees are typically deducted from the funds available in the investment fund, but may also be deducted in whole or in part from the payments made to the participants.

According to a second embodiment of the invention, there is provided a retirement product and inflation hedge for the provision of a stream of payments to a group of participant individuals fur an indefinite period associated with the longevity of the participants, the product comprising:

one or more computers or one or more computer servers, the one or more computers or one or more computer servers having a processor and memory storage with instructions which when executed by the processor perform predetermined functions;

a database stored on the memory storage of the at least one or more computers or one or more computer servers, the database being operable to receive information relating to a plurality of potential participants, the information including mortality criteria, the database having means for identifying, common mortality criteria and/or common expected mortality calculated from some or all of the mortality criteria relating to some or all of the potential participants thereby indicating a substantially equivalent mortality risk;

a definition function, performed by the one or more computers or one or more computer servers that defines from some or all the identified common mortality criteria and/or the common expected mortality, requirements for inclusion of potential participants in one or more substantially homogeneous mortality risk groups and thereby operable to identify a plurality of participants having a substantially equivalent mortality risk as determined by meeting the requirements for inclusion in the one or more groups;

one or more groups, operable to be formed from some or all of the identified participants, the matching of the participant in any group being operable and each assigned anonymous algorithms after providing biometric identifiers.

a group investment fund, operable to be funded with the aggregation of investment payments received from or on behalf of each participant in the group and operable to invest some or all of the investment fund in an asset allocation portfolio in accordance with predetermined investment guidelines intended to manage the investment fund; and

payment distribution means, operable to distribute a portion of the investment fund as payments at predetermined distribution periods, the portion being made available for distribution payments at the completion of any period being calculated according to a pre-defined actuarially fair calculation agreed between the participants; wherein in operation some or all of the longevity risk is operable to be transferred to the group of participants and an income stream of payments to the participants is made available for as long as they live or until there is only a predetermined number of survivors remaining from the original group or where a predefined time has elapsed after formation of the group or after the occurrence of another predefined circumstance.

According to a third aspect of the invention there is provided a system for the provision of a stream of payments to a group of participant individuals for an indefinite period associated with the longevity of the participants, the system comprising:

one or more computers or one or more computer servers, the one or more computers or one or more computer servers having a processor and memory storage with instructions which when executed by the processor perform predetermined functions;

a database stored on the memory storage of the at least one or more computers or one or more computer servers;

an information receiving function module, operable to receive information on the database relating to a plurality of potential participants, the information including mortality criteria, the database identifying common mortality criteria and/or common expected mortality calculated from some or all of the mortality criteria relating to some or all of the potential participants thereby indicating a substantially equivalent mortality risk;

a mortality assessment function module, per-formed by the one or more computers or one or more computer servers, and defining, from some or all the identified common mortality criteria and/or the common expected mortality, requirements for inclusion of potential participants in one or more substantially homogeneous mortality risk groups and thereby operable to identify a plurality of participants having a substantially equivalent mortality risk as determined by meeting the requirements for inclusion in the one or more groups;

a group formation function module, operable to form one or more groups from some or all of the identified participants, the matching of the participant in any group being operable to be selected by the participant or on behalf of the participant according to age cohort status;

an investment module, operable to form a group investment fund funded with the aggregation of investment payments received from or on behalf of each participant in the group and operable to invest some or all of the investment fund in an asset allocation portfolio in accordance with predetermined investment guidelines intended to grow the investment fund; and

a distribution module, operable to distribute a portion of the investment fund as payments at predetermined distribution periods, the portion being made available for distribution payments at the completion of any period being calculated according to a predefined actuarially fair calculation agreed between the participants;

an identification operation, from the information collected, that identifies common mortality criteria and/or common expected mortality calculated from some or all of the mortality criteria relating to some or all of the potential participants indicating a substantially equivalent mortality risk;

a definition operation, that defines, from some or all the identified common mortality criteria and/or the common expected mortality, requirements for inclusion of potential participants in one or more substantially homogeneous mortality risk groups;

an identification operation, identifies a plurality of participants having a substantially equivalent mortality risk as determined by meeting the requirements for inclusion in the one or more groups and collection of biometric data and algorithmic application making participants anonymous;

a group formation operation, that forms one or more groups of some or all of the identified participants, the matching of the participant in any group being selected by the participant or on behalf of the participant;

an investment operation, that establishes a group investment fund, the investment fund being the aggregation of investment payments received from or on behalf of each group participant

an investment operation, that invests some or all of the investment fund in an asset allocation portfolio in accordance with predetermined investment guidelines intended to grow the investment fund; and

a distribution operation, that after life-death verification process to determine survivors that distributes a portion of the investment fund as payments at predetermined distribution periods, the portion being made available for distribution payments at the completion of any period being calculated according to a predefined agreement, wherein some or all of the longevity risk is transferred to the group of participants and an income stream of payments to the participants is made available for as long as they live or until there is only a predetermined number of survivors remaining from the original group or where a predefined time has elapsed after formation of the group or after the occurrence of another predefined circumstance.

### BRIEF DESCRIPTION OF THE DRAWINGS

FIG. 1 shows a mortality distribution;

FIG. 2 shows by way of a Flow Chart the Administrative and enrollment processes along with the user interface;

FIG. 3 shows, by way of flow chart graphic representations of the life-death verification process relating to participants;

FIG. 4 shows by way of flow chart shows the Program Termination Process the Survival Sharing Pool in accordance pool type;

FIG. 5 shows examples of Pooled Capital Investments available under the Survival Sharing process;

FIG. 6 is a flow chart showing examples of the life cycle of cohort-based anonymous Investment pools utilized by way of the invention;

FIG. 7 is a chart of Mortality Distribution and life expectancy a age 0 and age 65

FIG. 8 is Social Security Actuarial Life Table, 2014, issued by the SSA; and

FIG. 9 provides examples of increasing yields to pool participants under embodiment s of this innovative invention;

The illustrations are intended to provide a general understanding of the concepts described and the structure of various embodiments, and they are not intended to serve as a complete description of all the elements and features of methods and systems that might make use of the structures or concepts described herein. Many other embodiments will be apparent to those of skill in the art upon reviewing the descrip-tion. Other embodiments may be utilized and derived there-from, such that structural and logical substitutions and changes may be made without departing from the scope of this disclosure.

It should also be appreciated that the figures are merely representational, and are not be drawn to scale and certain proportions thereof may be exaggerated, while others may be minimized. Accordingly, the specification and drawings, together with any examples, are to be regarded in an illustrative rather than a restrictive sense and the specific form and arrangement of the features shown and described are not to be understood or interpreted as limiting on the invention.

### DESCRIPTION OF EMBODIMENTS

In most developed countries, retirement planning is a growing concern, both from an individual and a societal point of view. The use of Defined Benefit plans (DB plans) has decreased significantly over the last few decades. Governments of developed countries around the world struggle with pressing and more noticeable issues such as financial system stability, health care, alarming deficits and high unemployment. In the private sector, firms struggle with aging work-force, high volatility of pension contribution and accounting, high pension administration cost and underfunded plans; which are all reasons that triggered the decline of DB plans in favor of the use of defined contribution plans (DC plans).

Retirement issues are not tackled as an urgent matter, leaving individuals with the burden of planning and providing for their own long term financial needs. This increasingly growing reality has opened up a world of both new challenges and new opportunities. Many individuals arc facing serious longevity risk, which is the risk of outliving one's assets. Many professional associations and organizations foresee longevity risk as a major concern in the current social, economic and demographic environment.

The need for individuals to manage longevity risk can be introduced by way of an illustrative hypothetical. As our population ages, how would a 65-year old widowed father of two, with $1,000,000 in assets, manage his retirement?. If he dies tomorrow, he will leave half a million dollars to each of his children. If the same man dies in 35 years and is confined to a nursing home for the last 10 years of his life, his two children may have to support him financially for several years. In retirement planning, a lot of importance is placed on variables such as diversification, risk, liquidity, fees and service. Surprisingly, very little emphasis is placed on the most important variable of all. How long we are going to live?

If this individual has no lifetime income, he will rely on his capital and investment gains to provide for himself. Even assuming the individual diversifies his asset portfolio to the maximum extent possible, picks stocks in a better way than the very best professionals can and does it all solely by himself without incurring any fees; the individual still does not know when he will die. What is the optimal investment strategy in order to provide for funds for the rest of one's life, when one does not know what that life span will be?

Further, should the amount any individual wishes to leave their heirs rely so heavily on the unknown variable of longevity?. By allocating a certain amount of savings to a life income product, one can exert a greater degree of control over the likely amount that may be left for inheritance. If our same individual has no life income, the difference between dying very early or very late can have a significant financial impact on his succession; as an early death will leave a substantial inheritance, while a late death may turn the parent into a liability for the children. If a certain part of savings could be converted to a life payment, the individual would depend less on his non-periodic portion of saving.

Individuals face a more serious longevity risk than their parents a generation ago; longevity risk is increasing and numerous factors appear to be shifting the risk to individuals, including the following:

Governments and corporations have altered their strategy relating to the assumption of large risks (longevity, economic, demographic or regulatory) and have shifted part of the burden back to individuals. In addition, alarming government debt levels coupled with advancements in technology (leading to longer life spans) have further emphasized the need for individual longevity protection.

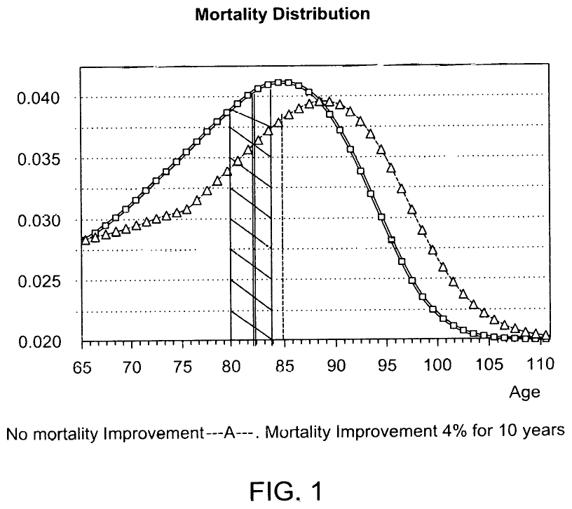

Individuals can expect to attain or exceed projected life expectancies easily; but as the expression implies, life expectancy is an expected value, which is nothing more than the weighted average of a distribution. Individuals remain unable to predict where they will on that distribution, i.e., when they will die. FIG. 1 shows such a mortality distribution. According to the “2007 Period Life Table for the Social Security”, a 65 year old male is expected to live to age 82, as illustrated with the dashed vertical line of FIG. 1. However, an expected value is not a certain indication of when that individual will actually die. In fact, according to the same assumptions used to determine this life expectancy, that same man (at age 65) has approximately only a 4% chaitee of dying at age 82. Further, that same individual has a 20% chance of dying in his early 80s (80 to 84), as illustrated by the hatched area on FIG. 1. In other words, our 65 year old man has a 96% chance of not dying at his life expectancy age (82 years old) and an 80% chance of not dying in his early 80s.

The American Academy of Actuaries (AAA) points out that over 30% of females aged 65 will live beyond age 90 (see N. Abkemeier, N. Bennett, D. Fuerst, T. Manning and T. Terry, “Lifetime Income: Risks and Solutions,” 2012). Put differently, if all women at age 65 plan to live up to age 90, 3 out of 10 will be in serious financial trouble; at a time when they will very unlikely be capable of going back to work. Also, the other women who died earlier could have deployed more capital earlier in their retirement, hence increasing their spending confidence level if they had somehow annuitized part of their funds as opposed to using a constant withdrawal method (such as the 4% rule-see, for example, C. O'Flinn and F. Schirripa, “Revisiting Retirement With-drawal Plans and their Historical Rates of Return,” O'Flinn Schirripa, 2010).

Further, life expectancy is likely to change over the course of a few decades;

With computer capabilities and technological advances reaching new levels every day, it is impossible to predict where technology will lead us. For example, in a US Time Magazine article about Gleevec (a drug manufactured by Novartis used to treat certain cancers), Dr. Larry Norton of Memorial Sloan-Kettering Cancer Center declared: “I think there is no question that the war on cancer is winnable” (see M. D. Lemonick, A. Park, D. Cray and C. Gorman, “New Hope for Cancer,” Time Magazine, 2001). Further, Bloomberg News reported in early 2012 that “The 30-year quest for an AIDS cure advanced as scientists succeeded for the first time in attacking HIV in its hardest-to-reach hideouts with a cancer drug made by Merck & Co” (see R. Langreth and S. Pettypiece, “AIDS Cure Quest Advances as Cancer Drug Finds Hidden HIV,” Bloomberg, 2012). These examples suggest a major medical breakthrough, such as a cure for a major disease or a successful preventive approach for a condition suffered by many, is possible in the medium term; resulting in a significant increase of life expectancy and the resulting increase of the burden on social security and medicare.

Retirement time span has increased in the last few decades; individuals not only live longer, hut also retire earlier, according to an Allianz working paper (see S. Benartzi, A. Previtero and R. H. Thaler, “Annuitization Puzzles,” Allianz, 2011). With people being retired for a longer period of time, it is clear that longevity risk is becoming increasingly more important.

The surrounding economic, social and demo-graphic environment coupled with governmental situations fuel speculation on future inflation paths in the decades to come. Central banks may succeed in fulfilling their usual key objective of maintaining inflation rates at a reasonable level, but other factors may overcome these efforts and push inflation higher than is economically desirable and conducive to growth.

Public debt in many countries has grown at an alarming rate and is now a global issue. Many fear that public sector undertakings made may not be supportable and that benefits like future pensions promised by governments may be compromised. In many ways, this invention can also be viewed as a way for governments to raise cheaper debt due to the added yield of the Mortality Credit to survivors, thus in fact creating a subsidy for government debt.

Three major insurance vehicles are currently available for addressing longevity risk: governmental benefits, defined benefit pension plans and guaranteed lifetime income products. Current trends suggest that Defined Benefit plans (DB plans) are being replaced with. Defined Contribution plans (DC plans). According to Towers Watson, in 1998, 90 of the Fortune 100 companies offered a DB plan. By 2010, that number had decreased to 42. Given these trends, it would perhaps be natural to expect a rise in demand for private lifetime income instruments. However, a recent Society of Actuaries paper suggests this is not the case: “Economic literature has nearly unanimously agreed that, at least from a pre-tax personal income perspective, the financial welfare of most retirees would be enhanced by annuitizing a substantial portion of their wealth. The lack of demand in the private annuity market has given rise to a large body of research that attempts to understand the aversion of individuals to annuitize.” See B. J. MacDonald, B. Jones, R. Morrison, R. Brown and M. Hardy, “Research and Reality-A Literature Review on Drawing Down Retirement Savings.,” Society of Actuaries, 2011). This aversion would effectively leave only governmental plans protecting individuals from longevity risk; a social mechanism where individuals have limited control. There are a number of shortcomings with each of the three alternatives, namely:

Governmental Plans

Many individuals rely on a governmental plan (such as Social Security in the US) as their main or supplemental source of lifetime income. However, there is considerable uncertainty regarding the future of these programs. In the US, the following sentence appears on individual social security statements: “Without changes, in 2033 the Social Security Trust Fund will be able to pay only about 75 cents for each dollar of scheduled benefits. We need to resolve these issues soon to make sure Social Security continues to provide a foundation of protection for future generations.” In order to fund this liability, the government will have to take intervening actions (fiscal or others) which pose a threat to the benefits that will be available future retirees. An additional weakness of governmental plans is the limited control afforded to individuals over their retirement planning and benefits, as the government unilaterally determines the benefits to which they are entitled. Furthermore, many individuals may need a way to manage any additional personal savings through retirement.

Defined Benefit Plans

Many factors explain the shift from DB to DC plans. The common explanation that DB plans are simply too expensive is not entirely consistent with the nuances of the situation. First, it is hard to compare the expenses of DB and DC plans on an apples-to-apples basis as the two structures are completely different. Second, it is unlikely that even the perception of expense would adequately explain the ubiquitous shift; pension benefits are part of compensation packages which are dictated by the laws of supply and demand. Both employers and employees have issues with DB plans and the ongoing migration from DB to DC plans is likely due to multiple factors including:

From an employer's perspective:

Volatility of contributions and accounting measurements creates uncertainty. Mortality and economic figures change greatly over time and can impact significantly the financial situations of both employees and employers, favorably or adversely. And

promised benefit payment that seems adequate for an individual at a given time may be insufficient or larger than necessary for the same individual a decade later, resulting in potential undesirable large profit swings.

Administration costs have increased. The

new age of employee mobility may have hastened the demise of pension plans. With a larger number of employees both joining and leaving a company every year, the cost of administering a pension plan have become far more expensive and laborious than it was when employee turnover was much smaller. Further, the increase in regulatory constraints has also contributed to the increase in administration costs in the past few decades.

Plans are underfunded. When pension plans are underfunded, the plan sponsor has to close the gap on the shortfall through a mix of contributions or excess returns. Increasing risk by having more growth assets introduces considerable financial volatility for the sponsor which is not generally desirable. On the other hand, making significant contributions may not be appealing in an economic downturn. As a result, many sponsors have decided either to close the plan to new hires or even freeze the plan to existing participants. This can have an effect on different generations of workers. It can also lead to multiple cost attributions problems, personnel issues, work conflicts and even, in some cases, bankruptcy.

Time horizon difference. Management's compensation and incentives time horizons are usually much shorter than pension guarantees' time horizons. This may put popularity, compensation and long term solvency into a conflicting triangle.

From an employee's perspective:

Lack of interest. Money down the road has limited value to people in general, especially when it is associated with having to stay employed with the same employer. Employees may be more likely to appreciate the value of an immediate salary raise than a pension augmentation, even if the value of the compensation for the latter is higher.

Portability. a few decades ago, it was not unusual to see employees work for one company for their entire career. As employment mobility has increased, a more portable pension benefit system has become desirable, if not necessary.

Inflation risk. As noted above, inflation risk is a serious consideration from the perspective of a newly retired worker starting to receive benefits. Very few DB plans are inflation adjusted in the US and while non-inflation adjusted DB plans guarantee a certain payment at a point in time, they do not necessarily guarantee that the value of the payment will meet the future costs of living.

Heterogeneous society. The typical DB plan was designed using the assumptions of a past generation and did not envision the myriad of ways in which the nuclear family has changed. It is normal that the needs for retirement change and adapt to meet different lifestyles and situations. Many segments of society are opting for plans which can accommodate the portability and flexibility that suits their individual situation.

Some solutions do currently exist in society to mitigate the risk of becoming a financial liability to dependents or the state. For example, various type of income annuities (variable and fixed) and other life guaranteed products are available in the market place and presented as potential solutions against longevity risk, but their sales have not yet reached close to the level needed to offset the decrease in DB pension plans. This may be explained by the following factors:

Expensive: insurance companies are in the business of managing risks. Those risks come at a cost. As noted by MacDonald et al: “Annuities are overpriced from an actuarial perspective in that the actuarial present value of the premiums is greater than the actuarial present value of the benefits (Mitchell et al., 1999; Orszag, 2000). This is owing to the insurer's administrative costs that are built into the premiums to cover marketing costs, corporate overhead, income taxes, regulatory compliance, contingency reserves, and profits, as well as the expensive mortality assumptions arising from adverse selection.” Drivers affecting cost include:

Reserves and capital. Annuities, just like all other insurance products, are largely regulated. Reserve and capital requirements imposed by regulators are usually very conservative in order to protect the general public. An insurance company must hold funds aside to ensure its ability to honor commitments under various contingencies (i.e. interest rate changes, mortality shifts, etc.). These assets usually return a lower rate than what investors in insurance companies require. So in order to maintain the return required by investors, insurance companies must embed the cost of reserving into policyholder premiums.

Reinsurance and securitization. Reinsurance and securitization transactions are often logical strategies for insurance companies to pursue. However, a transaction of this type of comes at a cost, which is ultimately transferred to the policyholder.

Selling expenses. The nature and complexity of annuities often require the involvement of an intermediary (a broker or an agent) which can add up to the ultimate cost needed to offer the product, which is assumed by the policyholder.

Profits. Shareholders of insurance companies demand a high return on their investment (annuity profit targets are usually in the 10-12% range (see N.

M. Kenneally and C. J. Bierschbach, “Measuring Profitability,” 2010; and M. R. Katcher and N. M. Kenneally, “Measuring and Improving Profitability,” in Session 57 PD, 2011) since they are assuming a significant risk. Ultimately, this load is passed on to the policyholder.

Anti-selection. The process for underwriting income annuities is often less strict than that for life protection products. While the longevity risk facing individuals in good health may be obvious, the longevity risk facing individuals in poor health cannot be ignored. This is important because the current products are an unattractive solution for the latter group since they are priced using assumptions for the entire pool with no differentiation. Impaired annuities have not yet reached a strong market, especially in North America where less than 12 carriers offered impaired annuities in 2005. This, in turn, triggers a vicious cycle where income annuities remain immovably priced for a healthy population, since only healthy individuals are motivated to buy the product under its current pricing structure.

Lack of transparency. The concept of an annuity is not easily grasped by a layperson and the terms of guarantees and fees are not always transparent. Variable annuities can be especially complex, as pointed out by Hube in Barron's: “Given a dizzying number of features and restrictions, contracts for some annuities-variable and otherwise”. And because each comes with its own small twists, these products can be very difficult to compare.” (see K. Hube, “Top 50 Annuities,” Barron's, 2012).

Mortality concerns. Compared to a traditional life insurance product, where customers usually assume the small payment of a premium in exchange for a large benefit, the early death of a policyholder can result in a significant financial loss in the case of a life guaranteed product. There is a risk that an individual may not benefit from his/her lifetime savings in case of early death just after annuitization.

Marketing challenges. While individuals are happy to benefit from employer-funded pensions, they may be less likely to purchase an income annuity of equivalent value, as it usually requires a significant financial commitment upfront and the risk of insolvency of the insurer.

The present invention relates to the concept of “Survival Sharing”, whereby small or large groups of anonymous individuals with similar mortality characteristics pool their investments together and receive distribution payments from that pool according to a set of predetermined and actuarially fair criteria. Survival Sharing shifts the investment and the mortality risk to the group of participating investors in a way that hedges each individual against longevity risk.

In recent years, a few academic papers have touched on how variations of the tontine concept could be adopted in modern times. One variation, described by Wadsworth et al. in “Reinventing Annuities,” Staple Inn Actuarial Society, 2001, is the annuitized fund. In his 2005 paper, Piggott builds a solid foundation for “group self-annuitization” and presents a robust mathematical model (see J. Piggott, E. A. Valdez and B. Detzel, “The Simple Analytics of a Pooled Annuity Fund,” 2005). Stamos builds on this model by discussing the optimization of consumption and asset allocation in the context of pooled annuity funds (see M. Stamos, “Optimal Consumption and Portfolio Choice for Pooled Annuity Funds,” 2007). Piggott's model was further refined by Qiao et al., who pro-posed adding a systematic mortality risk component (see C. Qiao and M. Sherris, “Managing Systematic Mortality Risk with Group Self Pooling and Annuitization Schemes,” Australian School of Business Research Paper No. 2011ACTL06, 2011). In Goldsticker's 2007 paper, he introduces a practical tontine variation where shares of deceased members are separated among survivors (see R. Goldsticker, “A mutual fund to yield Annuity-like Benefits,” Financial Analyst Journal, a CFA publication, Volume 63, 2007). Baker et al. proposes the tontine concept in a non-longevity context: health coverage for young adults (see T. Baker and P. Siegel-man, “Tontines for the Young Invincibles,” REGULATION, WINTER 2009-2010). In his 2009 working paper, Rotemberg proposes a variation of tontines called a Mutual Inheritance Fund (MIF) where shares of deceased members are separated among surviving members, enabling an increasing payoff between survivors through time (see J. J. Rotemberg, “Can a Continuously-Liquidating Tontine (or Mutual Inheritance Fund). Succeed where Immediate Annuities Have Floundered?,” Working Paper, 2009). His model differs from Gold-sticker's by proposing increasing distributions, as opposed to annuity-like benefits. Sabin introduces yet another variation called the Fair Tontine Annuity (FTA), where he presents, among other things, a sophisticated algorithm to allocate deceased tontine members' shares back to surviving members (see M. J. Sabin, “Fair Tontine Annuity,” 2010). These ideas share some similarities with each other and with the present invention, while the present invention simultaneously maintains several elements of distinctiveness, including maintain the anononimity of pool participants through the use of biometric identifiers, algorithms, hyper-ledgering and block chain applications.

The present invention consists of forming homogeneous groups of a sizable number of individuals possessing similar characteristics (such as the same age, gender, health condition, etc.); which characteristics relate to mortality criteria and/or enable determination of expected mortality from the mortality criteria. Each participant chooses his investment medium and is pooled and contributes capital to the pool group participant then contributes the same amount to an investment fund. Similar to a traditional annuity, the present invention generally includes two phases: the accumulation phase and the distribution phase. The investment fund typically includes an asset allocation portfolio, which is invested and reinvested with a view to the generation of investment returns in the accumulation phase, in a manner analogous to a mutual fund. At a predetermined date, after an accumulation period of the accumulation phase, the investment fund is redistributed in an actuarially fair way to each surviving participant investor in small installments over time, mimicking an income annuity payment benefit. Typically, at each period, the administrator determines the survivors of the pool entitled to distributions or dividends and payments are recalibrated and distributed among the survivors of the group.

The Survival Sharing cycle differs from that of a traditional insurance product in that the latter is usually priced and sold before entering an iterative process where claims are paid and reserves established. In contrast, Survival Sharing pools investments of individuals before entering its iterative process of redistributing the fund in an actuarially-fair manner according to the terms of the pooling agreement or criteria.

Among other roles, the provider or administrator acts as an independent redistributor of the fund. The goal of the administrator is to validate life-death and to keep payments to survivors as well as wind up when the particular pool or programs terminates under their specific criteria.

Each surviving member is expected to receive a series of payments, but since the group of investors is assuming the mortality and investment risk, the distribution will vary over time in ever increasing amounts as mortality

takes its toll;

Group size is relevant (a high number of participants decreases the sample risk for each individual), but substantial group mortality homogeneity is of even greater importance in maintaining the overall fairness of concept. Individuals have the availability to reduce sample risk by purchasing multiple shares of groups formed and operated in accordance with the present invention on their own timeline.

Example: One year (see section “calculation details” for assumptions)

In a first example, 10 male participants each con-tribute $10,000 at age 45 (at t=0) and agree to receive distributions from the fund at age 65 (at t=20). No deaths or surrenders occur during the accumulation phase and the net annual return is 4.6880235%. After 20 years, the initial fund of $100,000 has grown to $250,000 and the initial annual distribution per member at age 65 is $2,148. The distribution amounts will be recalculated periodically, taking into account variations in mortality experience and expectations, investment returns and economic environment. Assuming the rate of return and assumed interest rates remain unchanged in the following year, the fund will now have a value of $239,237 at age 66. Scenario #1 assumes no participant will die during this year. The resulting balance is split among the ten individuals, which results in a distribution payment of $2,114 per member at age 66 (at t=21). Scenario #2 assumes the same investment returns, but this time one participant has died during the year; the balance is split among the nine remaining individuals, which results in a distribution payment of $2,349 per member at the age of 66 (at t=21). The member who passed away exits the group and will no longer receive distributions. The following table summarizes the payments after one distribution year.

| Time | Age | Feature | Scenario #1 | Scenario #2 |
|---|---|---|---|---|
| t- 0 | age - 45 | Fund Value | $100.00 | $100,000 |
| t- 20 | age - 65 | Fund Value | $250.00 | $250,000 |
| | | Survivors | 10 | 10 |
| | | Payment | $2,148 | $2,148 |
| t- 21 | age - 66 | Fund Value | $239.23 | $239,237 |
| | | Survivors | 10 | 9 |
| | | Payment | $2,114 | $2,349 |

This process transfers the investment and the mortality risks to the entire group. This example is used for illustrative purposes; in a real situation, mortality would be reevaluated, interest rates would be reassessed, a fee for service might be deducted from the account value and multiple other provisions can be made available to investors at a fee.

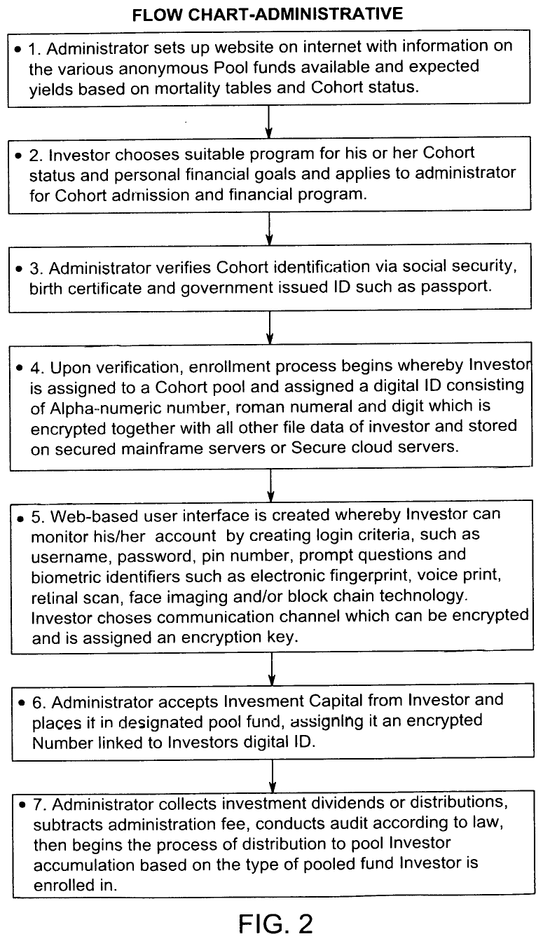

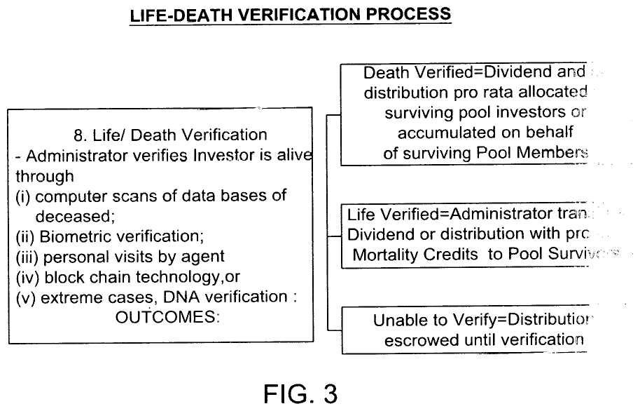

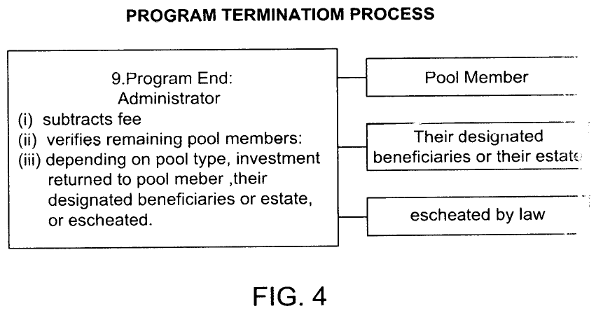

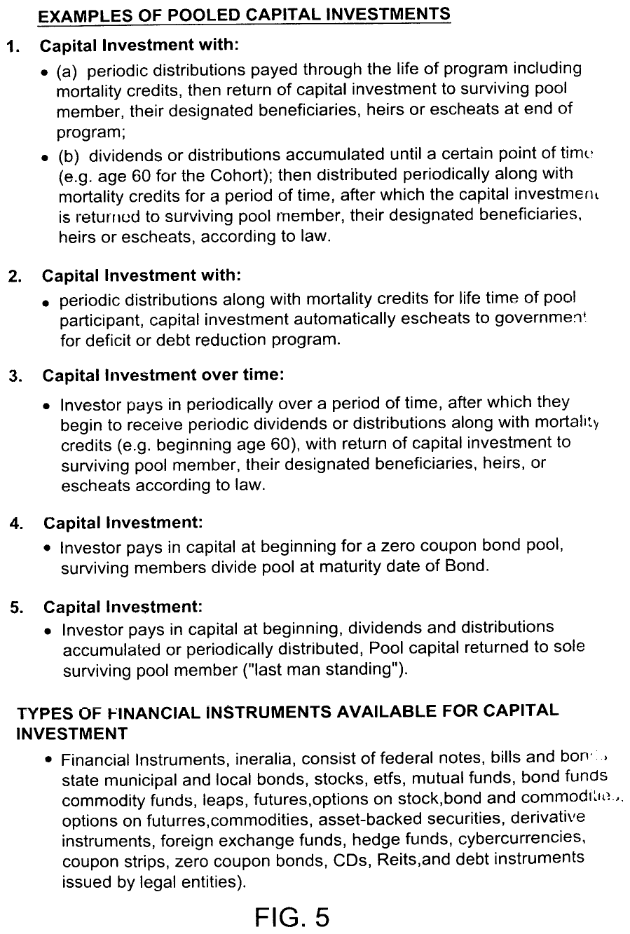

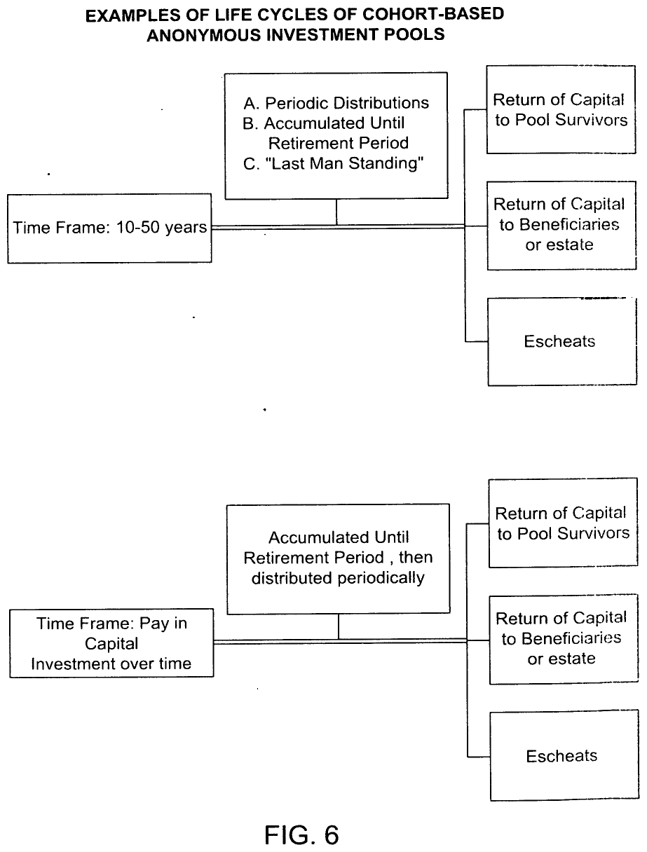

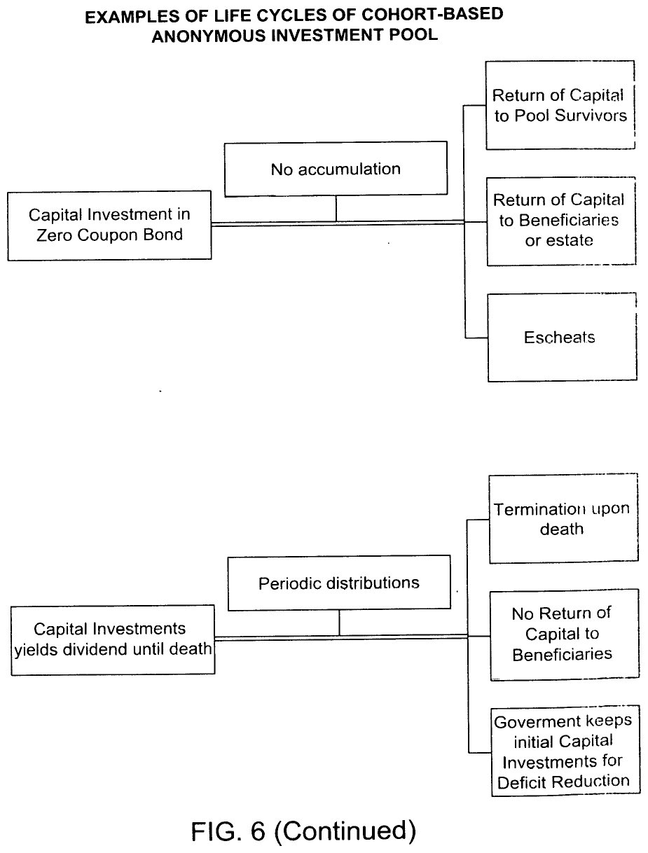

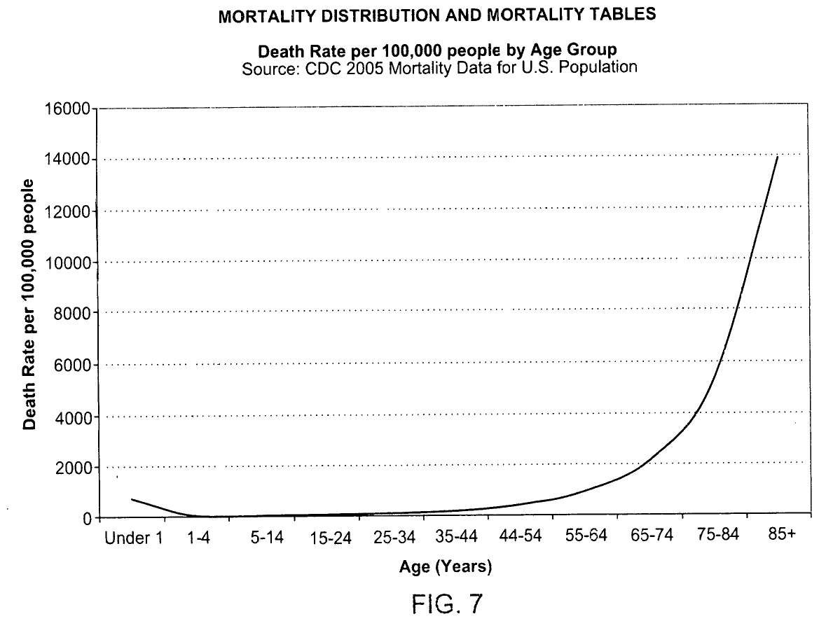

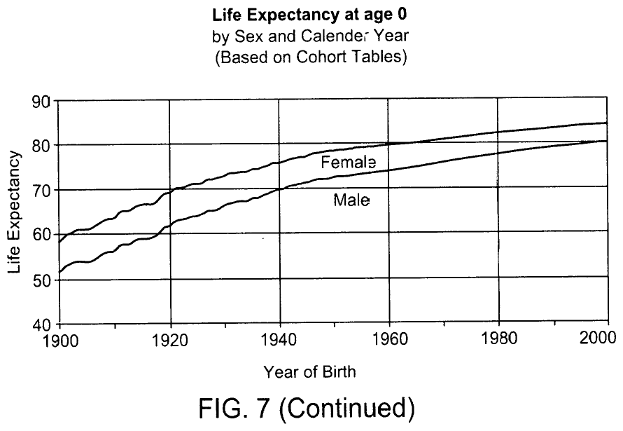

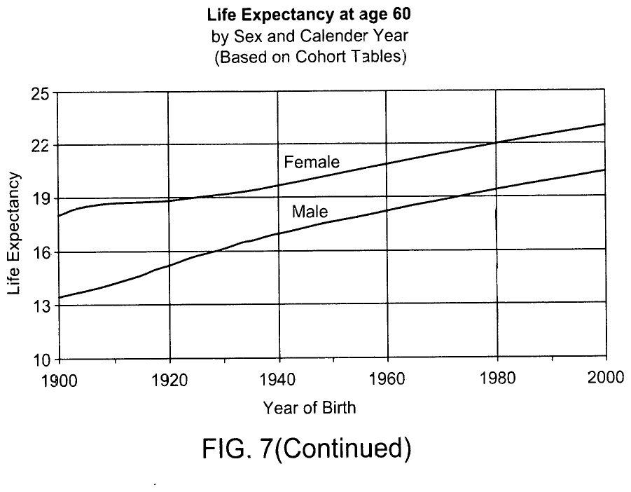

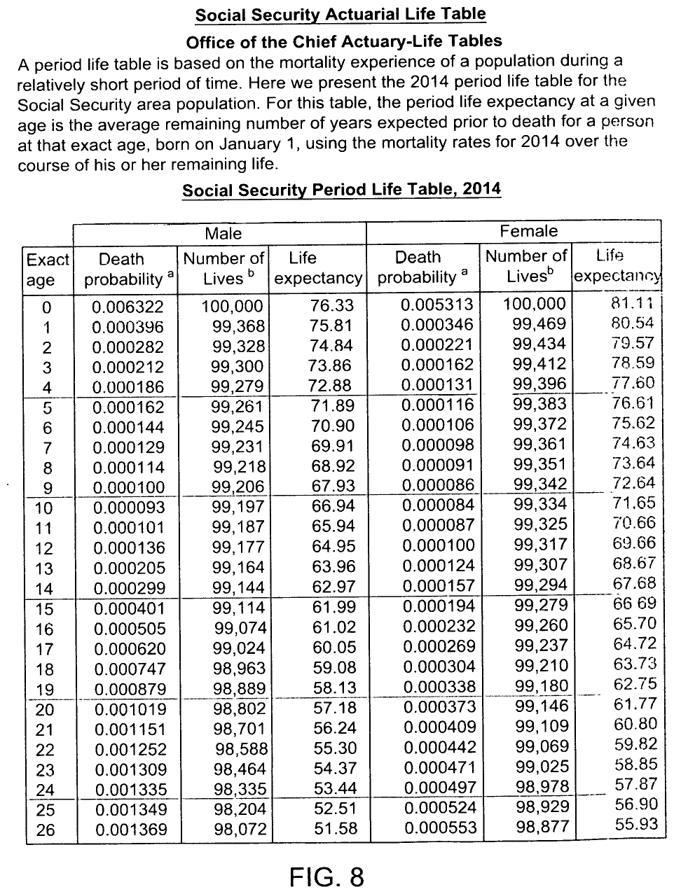

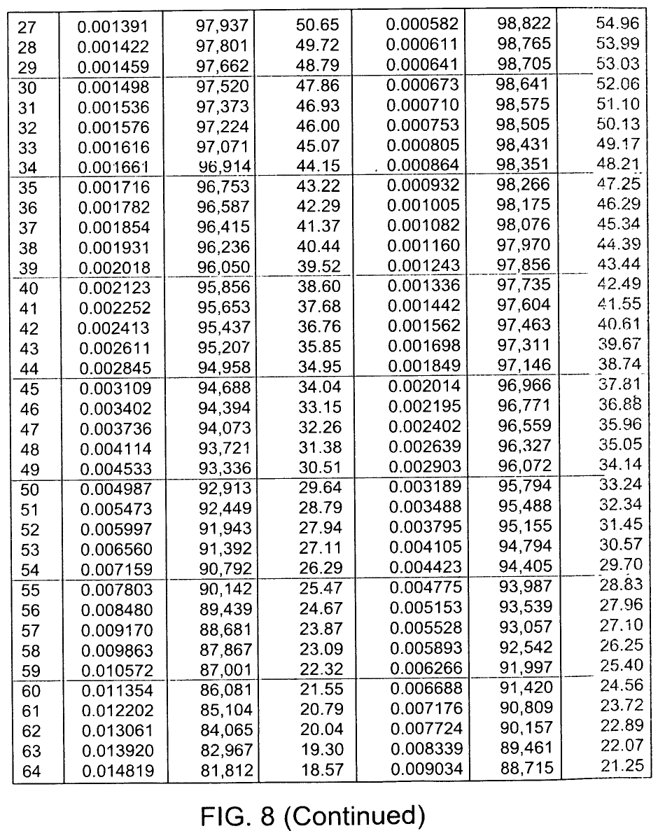

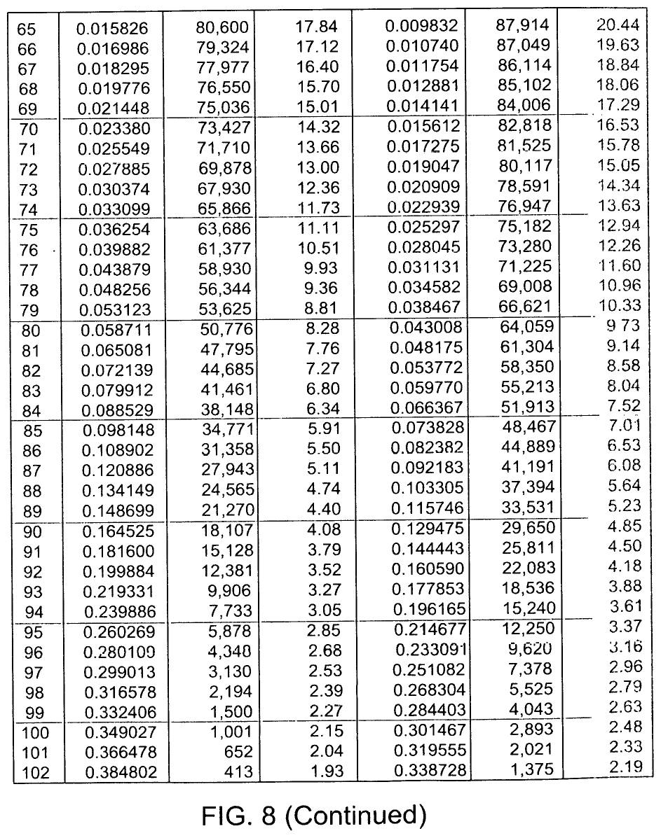

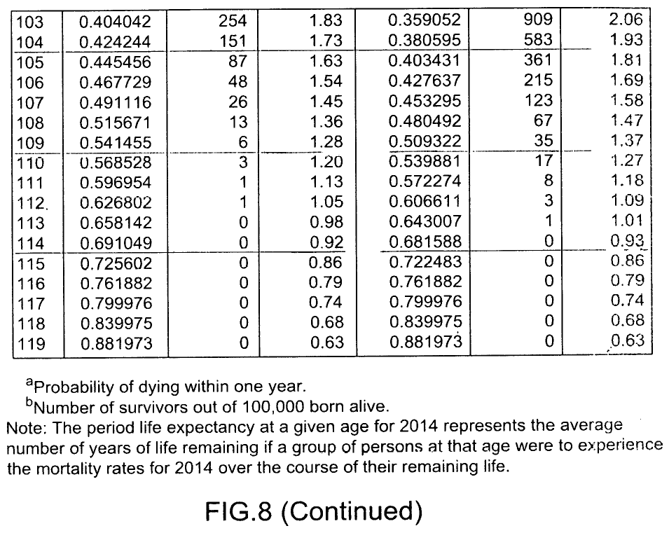

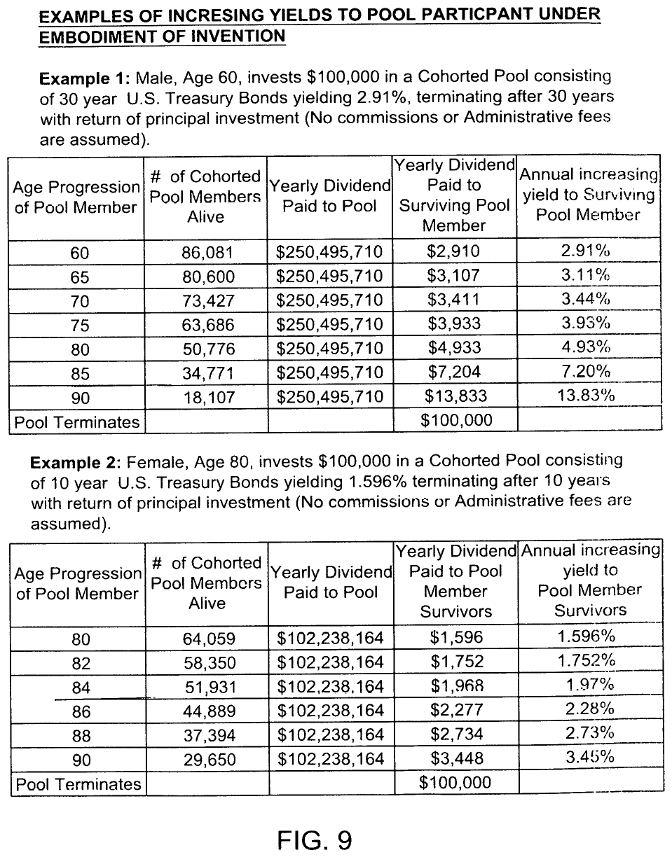

FIGS. 2-6 show the steps that maybe undertaken in the practicing of the present invention. In step A an individual realizes the need for longevity and typically establishes contact with a Survival Sharing provider. It will be appreciated that the provider may undertake certain marketing activity to promote Survival Sharing. In step B, a triage phase leading to group establishment, the individual inputs his/her objective personal characteristics (into an information receiving module), including certain mortality criteria (information that may be used in assessing the individual's personal expected mortality), including the date of birth (age), gender, medical history and independent diagnosis (made by professional third party and/or by an electronic system algorithm). Additional information such as job type, participation in sports and activities, personal habits (smoking, drug use etc.), may be considered mortality criteria. The provider gathers the data collected and results generated from information collected (such as expected mortality determined in whole or in part from mortality criteria) and stores this all in a readily available database.

In step C, group selection, the individual chooses a group from the available option features and the provider allocates the individual into a group. Given the data provided in step B, a software program is typically used (in the information receiving module) to identify common mortality criteria and/or common expected mortality of potentially par ticipating individuals who have provided information. From this, in a mortality assessment module, requirements for inclusion in one or more groups (assessed as having substantially comparable homogenous mortality risks) are defined. Using a group formation module, groups that may be avail-able to the individual (meeting the requirements) are then identified and displayed to the individual for selection,. Generally, the provider software generates standard options with standard features from the available groups, or conversely, the individual can input his/her preferred features and the software can suggest a close matches according to those preferences.

The individual then provides biometric data such as retina scans, electronic fingerprints, facial scans, voice prints and is provided with algorithmic keys to make them anonymous to fellow pool participants while providing the means to interface with the administrator.

A group is then formed with a plurality of appropriately categorized individuals.

In step D, contribution, each participating individual makes a payment into an investment fund formed in an

[symbol]

Survival Sharing investment/distribution offerings of the present invention are characterized by a wide variety of flexible features including:

Investment assets. Investors among a pool need to agree on a predetermined asset allocation portfolio. The risk/reward preferences are determined by the individual.

Distribution age. While the current age may be fixed, the age at which distributions commence is left for the individual to decide based on pool criteria. It will be appreciated that numerous variations of the present invention are contemplated as being within the spirit of the general concepts outlined, whether in the field o retirement planning or in other applications. Investment module (typically investing some or all of the proceeds into an asset allocation portfolio for investment in accordance with agreed guidelines or specifications.

There invention and presently available retirement are numerous differences between the present vehicles

Survival Sharing differs from a DB pension plan or life guarantee products which are vehicles locking in a periodic payment.

[symbol]

In contrast, Survival Sharing does not lock in a fixed distribution payment and does not incur any liability. It pools investments of homogeneous investors together and redistributes the fund in the smoothest possible way to survivors in a periodic manner. Survival Sharing transfers the longevity risk to the pod of participants. The cost from the provider's perspective is therefore significantly reduced and translates into an expected benefits increase for the investors. Similar to variable annuities, Survival Sharing offers different possible mixes of assets, enabling a higher risk reward option.

Compared to many insurance products, Survival Sharing is more fairly manageable. Features are generally negotiated virtually and anonymously between each member of a group, as opposed to incurring a charge or a penalty for a particular option. This virtual negotiation takes the shape of a supply and demand mechanism, so individuals do not actually meet and negotiate in a traditional manner. If pools with a certain feature (e.g. return of capital to a non-survivor beneficiary) are sought out more than others, in time they will become more readily available. Investors who prefer other features may be forced to compromise on their preferences or on group sizes, but these features will be shared among individuals and not structured in a fee or penalty as they are for insurance products. For example, a deferred annuity charging a high surrender charge should generate a higher return in comparison to another annuity charging a smaller surrender charge, all else being equal. This is often difficult to measure, predict or compare. With Survival Sharing, an individual can pick and choose its group and features, as long as personal characteristics are met and other people want to be in the same group.

Advantages of the present invention include:

For Individuals

Effective for managing longevity risk

Longevity risk requires a grouping mechanism (pooling, hedging, insuring etc.) in order to function. Survival Sharing introduces an innovative way for individuals to manage longevity risk on their own. Further, by using Survival Sharing, one can be in a better position to control desired bequest amounts and create an estate that does not depend on their longevity.

Survival Sharing may also be attractive when analyzing the added risk assumed by the investor relative to the added return generated for an individual already facing investment risk; the increase in volatility in the position taken by assuming some longevity risk is less than the increase in return, all else being equal since longevity risk is uncorrelated to investment risk.

Cost-effective

Since Survival Sharing does not secure a guarantee, many costs associated with the said guarantee are saved and, consequently, passed back to the investor. Sabin, for example, estimates “that insurers typically charge a premium that is 14% higher than fair.” Sabin uses the word “fair” in a statistical way where the 14% represents the bias between what the insurer is charging for contract and the purely statistical position the insurer is assuming on the same contract; the 14% difference is essentially a load for profit and expenses, which is ultimately paid by policyholders.

Suitable for a significant portion of the population Survival Sharing is a product that is suitable for individuals possessing a significant portion of their assets in non-life income vehicles.

Accessible at a fair price for all

Traditional guaranteed income products are often priced for healthy lives. As discussed, very few carriers offer impaired annuities for individuals in poor health. Since longevity risk is a concern for all, Survival Sharing allows formation of groups of similar health, making it fair among participants.

Furthermore, Survival Sharing allows if not mandates participation anonymously.

Tailored and user-defined

Survival Sharing enables a fair, dynamic and flexible feature-selection process. Each feature is ultimately shared equivalently between individuals and each group member can participate on either side of the equation.

Survival Sharing distribution payments may vary through time, but the bylaws dictating those payments remain consistent, transparent and uniform through each contract. Traditional insurance products are often subject to modifications and fine-print disclaimers.

Removes all or nothing amount dilemma

Annuities are usually sold in large premium amounts which add to the daunting experience of their purchase. Survival Sharing allows smaller installments to individuals, making it smoother and allowing the buyer to become familiar with the product before making a larger commitment.

Removes all or nothing longevity risk

Individuals currently face two choices when dealing with longevity risk: lock into an income annuity or shoulder the full risk. Survival Sharing offers a balance between the two extremes. With Survival Sharing, one can now generate lifetime income and control their estate planning without worrying about their unknown date of death.

Flexible to the risk appetite of the individual

Survival Sharing enables individuals the flexibility to take on more risks with their savings, hence potentially generating higher returns in the long run.

Natural long-term care and medical costs hedge

As individuals live longer, health care and long-term care costs are likely to increase. Survival Sharing is not meant to be a substitute for long-term care, health or disability insurance, but does pro-vide income at a critical time for certain individuals.

Effective for hedging interest risk

Large interest rate swings can have a huge impact on income revenues. Survival Sharing can deploy capital progressively over a lifespan, and thereby smoothens the process.

No provider default risk

Although rare, insurer default is not impossible. Survival Sharing limits default risk to the asset exposure desired.

Empowers and encourages decision making from a position of strength

In the past, retirees have often had to wrestle with tough decisions at a time when it was least convenient and judgment could be heavily compromised. Survival Sharing enables more individuals to make informed decisions while they are still in a position of strength and confidence.

For Society:

Provides a financial incentive to stay healthy

Survival Sharing incentivizes individuals to make healthy choices, since the longer they live, the greater their financial benefit.

Reduces intergenerational conflicts

With Survival Sharing, no segment of society is on the hook. Each group is self-sustainable and assets are redistributed by an independent party.

Addresses growing concern of longevity risk

Longevity risk is no longer a fringe issue for many organizations. The American Academy of Actuaries has put together the “Lifetime Income Risk Task Force” in order “to address the risks and related issues of inadequate lifetime income among retirees”. Similarly, The International Monetary Fund dedicated a whole chapter to “The Financial Impact of Longevity Risk”. As lifespans increase, longevity risk will be a growing topic of interest and concern for a wide spectrum of organizations.

Increases distribution channels for retirement products

To date, the Internet sales of annuity products have not matched the comparable expected levels of other societal goods and services. Survival Sharing is designed to be accessible in a fashion that suits customers of the 21^{st} century.

Decreases governmental role in issues of retirement

In the past, individuals have often taken for granted a magical age of retirement (e.g., age 65). This has often created political conflict, especially at times when governmental solvency has been turbulent. Survival Sharing shifts the timing vs. reward dilemma choice to each individual and thus reduces a significant layer of politics and the need to explain possible unpopular decisions (e.g., increasing the retirement age to ensure pension fund solvency). Survival Sharing also increases societal awareness of the multiple entities and how they interact.

Reduces need for outside regulation

The need for outside regulation is decreased since the pool interest is a security and governed by state and federal securities laws;

Empowers and educates society about retirement planning.

Survival Sharing

empowers each user to play a hands-on role in their retirement planning and make decisions regarding ideal retirement age and target accumulation. A society that is better educated on the aspects of retirement planning is likely to be more financially stable. positive and productive.

It is believed Survival Sharing positions itself distinctly by offering a product that allows investors to keep more of their invested funds, while significantly decreasing their longevity risk. Survival Sharing is designed to be bought progressively, as opposed to an all or nothing product. This allows individuals to slowly build their retirement portfolio with shares in other additional groups over the years and become familiar with Survival Sharing and its features while diversifying investments returns and mortality risks along the way. Survival Sharing can also supplement other forms of retirement vehicles.

### Additional General Statements

Reference is made in this specification to the application of computers, computer implemented systems and computerized modules, that may be used in accordance with the method and system of the embodiments of the present invention and as applied in some example embodiments. It should be appreciated that a set of instructions may be executed, for causing such machines, systems and/or modules to perform any or more of the methodologies discussed above. The machine may operate as a standalone device or maybe connected (e.g. networked) to other machines. In a networked deployment, the machine may operate as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while a single machine may be described, a single machine shall also be taken to include any collection of machines that individually or jointly execute a set (or multiple sets) of instructions to perform any one or more of the methodologies or functions described in this specification. In addition, the growth of hyper ledgering and block chain technologies are evolving to where applications could be useful and cost-effective.

Machine-readable media may be provided, on which is stored one or more sets of instructions (e.g., soft-ware, firmware, or a combination thereof) embodying any one or more of the methodologies or functions described in this specification. The instructions may also reside, completely or at least partially, within the main memory, the static memory, and/or within the processor during execution thereof by the computer system. The instructions may further be transmitted or received over a network via the network interface device.

In example embodiments, a computer system (e.g., a standalone, client or server computer system) configured by an application may constitute a “module” that is configured and operates to perform certain operations. In other embodiments, the “module” may be implemented mechanically or electronically; so a module may comprise dedicated circuitry or logic that is permanently configured (e.g., within a special-purpose processor) to perform certain operations. A module may also comprise programmable logic or circuitry (e.g., as encompassed within a general-purpose processor or other programmable processor) that is temporarily configured by software to perform certain operations. It will be appreciated that the decision to implement a module mechanically, in the dedicated and permanently configured circuitry, or in temporarily configured circuitry (e.g. configured by software) may be driven by cost and time considerations. Accordingly, the term “module” should be understood to encompass a tangible entity, be that an entity that is physically constructed, permanently configured (e.g., hardwired) or temporarily configured (e.g., programmed) to operate in a certain manner and/or to perform certain operations described herein.

The term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies or functions in the present description. The term “machine-readable medium” shall accordingly be taken to include, but not be limited to, solid-state memories, and optical and magnetic media.

1. A life-death verification process wherein the life or death of the pool participant is confirmed, and;
distributing a portion of the investment fund to surviving pool members as payments at predetermined distribution periods, for as long as they live or until there is only a predetermined number of survivors remaining from the original group or where a predefined time has elapsed after formation of the group or after the occurrence of another predefined circumstance.

2. “the method of claim 1, wherein the mortality criteria relates to the potential participants age, gender, and health condition.

3. The method of claim 1, wherein the mortality criteria relates to the potential participants' age, gender and health condition and may additionally include criteria considered relevant to expected mortality, including living habits, habitat, and type of employment.

4. The method of claim 1, further comprising an accumulation phase, wherein the asset allocation portfolio is invested for a predetermined accumulation period prior to the initiation of the distribution phase.

5. The method of claim 1, further comprising an accumulation phase, wherein the asset allocation portfolio is invested for a predetermined accumulation period prior to the initiation of the distribution phase and the participants are afforded the right to leave the group voluntarily at any time during the accumulation phase.

6. The method of claim 1, further comprising an accumulation phase, wherein the asset allocation portfolio is invested for a predetermined accumulation period prior to the initiation of the distribution phase, and wherein in the distribution phase payments are made only to any surviving participants remaining or, where the participants have agreed in advance, that the payments be made to survivors and to beneficiaries of survivors for a predetermined initial period after the accumulation period or upon program termination.

7. The method of claim 1, wherein the predefined actuarially fair calculation accounts for variations in the investment results achieved, in anticipated future investment returns, in the group's mortality experience and in expected future mortality risk.

8. The method of claim 1, wherein the income stream of payments is made after the effluxion of predefined time, the time being determined based on the age of the group or the period since group formation. Claim 9. The method of claim 1, wherein investment fees are deducted from the funds available in the investment fund, or deducted in whole or in part from the payments made to the participants.

10. an information receiving process to receive information on the database relating to a plurality of potential participants, the information including mortality criteria, the database identifying common mortality criteria and/or common expected mortality calculated from some or all of the mortality criteria relating to some or all of the potential participants thereby indicating a substantially equivalent mortality risk;
a mortality assessment process performed by the one or more computers or one or more computer servers, and defining, from some or all the identified common mortality, criteria and/or the common expected mortality, requirements for inclusion of potential participants in one or more substantially homogeneous groups r
a group formation process, operable to form one or more groups from some or all of the identified but anonymous participants,
an investment module process to form a group investment fund funded with the aggregation of investment payments received from or on behalf of each participant in the group and operable to invest some or all of the investment fund in an asset allocation portfolio in accordance with predetermined investment guidelines; and;
a module life-death verification confirmation process for pool participants, whereby if there is an absence of routine timely participant check in for “proof of life” with electronic fingerprint, retinal scan, facial scan, voice print, etc. there then is human intervention that follows a process: letter, phone call, email, if there continues to be an absence of timely response, a visit possibly with a DNA test which is the only human intervention in the Administrator for final determination of pool shrinkage and dividend or distribution entitlement. Their entitlements to increasing yield investment returns are accounted for through block chain and hyper ledgering.
Further to an automated life-death verification process, the computers are asleep and are awakened to function only when they receive an encrypted signal that “wakens” them and initiates their functionality. This process begins with webcrawlers, which scour the web for death certificates or death notices, gathering such, dump such data into the data cache of a stand alone “Identifier Computer” which awakens and matches the data in the cache of all dead people against markers in its secure data base consisting of name, address, age, social security number of pool members.
a module process where Identifier Computer finds a possible match from the data cache provided by the webcrawlers, it then sends an encrypted signal a stand alone “Life-Death Verification Computer” to awaken. The Life-Death Verification computer does exactly that, it unwraps the subject file wrapper containing the biometric data, then proceeds to verify the life or death of the subject by means of electronic communication and biometric confirmation and/or human intervention. If the subject is verified as alive, the file is rewrapped and stored and the computer goes back to sleep. If death is verified, the computer sends an encrypted signal to the Administrator computer which subtracts the subject's distribution and allocates it prorated amongst remaining living pool members.
Their entitlements to increasing yield investment returns are again accounted for through block chain and hyper ledgering.
a distribution module process, operable to distribute a portion of the investment fund as payments at predetermined distribution periods, the portion being made available for distribution payments at the completion of any period:
wherein in the operation an income stream of payments to the participants is made available for as long as they live or until there is only a predetermined number of survivors remaining from the original group or where a predefined time has elapsed after formation of the group or after the occurrence of another prrdefined circumstance or program termination;
and wherein and whereby fees are allocated and the program is terminated by the administrator, wound down, and all predetermined criteria fulfilled.

11. (canceled)
